SEQUENCE LISTING (4 sequences)

SEQ ID NO: 1 - DNA, 19 bp, synthetic construct
  misc_feature 1..19: note = CD36 forward primer
  source 1..19: mol_type = other DNA
aatggcacagacgcagcct

SEQ ID NO: 2 - DNA, 18 bp, synthetic construct
  misc_feature 1..18: note = CD36 reverse primer
  source 1..18: mol_type = other DNA
ggttgtctggattctgga

SEQ ID NO: 3 - DNA, 22 bp, synthetic construct
  misc_feature 1..22: note = Gapdh forward primer
  source 1..22: mol_type = other DNA
agtatgactccactcacggcaa

SEQ ID NO: 4 - DNA, 20 bp, synthetic construct
  misc_feature 1..20: note = Gapdh reverse primer
  source 1..20: mol_type = other DNA
tctcgctcctggaagatggt

(19) United States
(12) Patent Application Publication
(10) Pub. No.: US 2025/0288554 A1
(43) Pub. Date: Sep. 18, 2025

(54) PROPHYLACTIC OR THERAPEUTIC AGENT FOR NON-ALCOHOLIC STEATOHEPATITIS-DERIVED HEPATOCARCINOMA

(71) Applicant: SEAKNIT Biological Technology Co., Ltd., Chendu, Sichuan (CN)

(72) Inventors: Takayoshi SUGANAMI, Nagoya (JP); Miyako TANAKA, Nagoya (JP); Noriko ASAHARA, Kyoto (JP); Hideo NISHIMURA, Kobe (JP)

(73) Assignee: SEAKNIT Biological Technology Co., Ltd., Chendu, Sichuan (CN)

(21) Appl. No.: 18/878,543

(22) PCT Filed: Jun. 22, 2023
(86) PCT No.: PCT/JP2023/023107

§ 371 Date: Dec. 23, 2024

(30) Foreign Application Priority Data
Jun. 23, 2022 (JP) 2022-100812

(51) Int. Cl. A61K 31/352; A61P 35/00

(52) U.S. Cl. CPC A61K 31/352; A61P 35/00

(57) ABSTRACT

The purpose of the present invention is to provide a prophylactic, therapeutic or ameliorating drug or food for non-alcoholic steatohepatitis-derived hepatocarcinoma. The present invention pertains to a CD36 inhibitor comprising taxifolin or a salt thereof. The present invention also pertains to a prophylactic, therapeutic or ameliorating drug or food composition for non-alcoholic steatohepatitis-derived hepatocarcinoma.

### TECHNICAL FIELD

The present invention pertains to a CD36 inhibitor comprising taxifolin or a pharmacologically acceptable salt thereof, as well as to a prophylactic, therapeutic, and/or ameliorating drug or food containing it for a disease pertaining to activation of CD36, especially for nonalcoholic steatohepatitis-derived hepatocarcinoma.

### BACKGROUND ART

CD36, which is a scavenger receptor to capture a fatty acid, has been proposed as a prognosis marker for various cancers originated mainly from the epithelium (breast, prostate, ovary, intestines), as well as hepatocarcinoma and glioma. Examples of CD36 function include to facilitate capturing of a fatty acid into a cell (translocase) thereby removing an adhesion receptor (NPL 1), to activate a glycolysis system by a cancer cell (Warburg effect) (NPL 2), to induce migration and metastasis of a cancer cell (NPL 3), and to facilitate a scaffold-independent propagation (NPL 4); thus, it is suggested that to prohibit the function of CD36 is effective to suppress the metastasis of a cancer cell.

However, the clinical trials using an antibody that targets CD36 have demonstrated that this is not effective on therapy of the metastasis of a cancer cell, suggesting difficulty in the use of CD36 as a target (NPL 5). Accordingly, it is desired to develop the method for inhibiting the CD36 activity, which is different from the use of the anti-CD36 antibody that directly acts to CD36.

Taxifolin is the compound represented by the following formula (1):

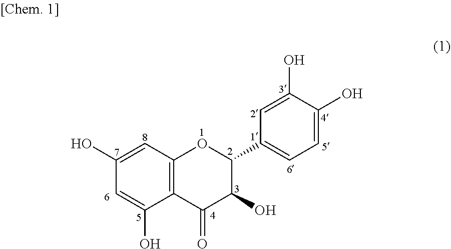 [SMILES: O=C1c2c(O)cc(O)cc2O[C@H](c2ccc(O)c(O)c2)[C@H]1O]

This is an extracted substance from a wooden portion of Dahurian larch and of Siberia larch. Recently, the ingestion safety of taxifolin has been confirmed (NPL 6), and also many actions of taxifolin have been known, which include an antioxidant effect in a model mouse suffered from acute pancreatitis (NPL 7), an amelioration effect of blood glucose in a model mouse suffered from the type 1 diabetes (NPL 8), a suppression effect of amyloid B agglutination in a model mouse suffered from Alzheimer's disease, an ameliorating effect of cerebral blood flow, an ameliorating effect of dementia (PTL 1 and NPL 9), among others. Apart from these, the inventors of the present invention found that taxifolin is effective to prevent nonalcoholic steatohepatitis (hereinafter this is referred to as “NASH”), among other things (PTL 2).

Due to the westernization of eating habits and lack of exercise, obesity has been increased in Japan, leading to the increase in the population of diabetes mellitus and of metabolic syndrome including the potential patients thereof to 22 million and 20 million, respectively. Moreover, due to the increase in obesity population, patients of the nonalcoholic fatty liver disease (NAFLD) including NASH are rapidly increasing; it is estimated to reach 15 to 20 million, which is about 30% of those who had visited a hospital for a medical check. According to the most recent data, about 25% of the population thereof progresses to NASH; and it is estimated that about 1/16 of the NASH patients will develop hepatocarcinoma after 10 years. However, so far there have been no reports at all on the taxifolin's inhibition action on CD36 and the suppression effect thereof on the onset of hepatocarcinoma in the patients who have been suffered from NASH, which has a risk of developing hepatocarcinoma.

### CITATION LIST

### Patent Literatures

[PTL 1] WO 2017/199755 A1
[PTL 2] WO 2020/262703 A1

### Non Patent Literatures

[NPL 1] J. Lipid Res. 2018; 59:1084-1093
[NPL 2] Cell Death and Disease, 2021; 12:328-341
[NPL 3] Frontiers in Genetics, 2019; 10: Article 680
[NPL 4] Oncogene. 2018; 37(17): 2285-2301
[NPL 5] BioMed Res Int. 2018; Article ID 7801202
[NPL 6] Int. J. Toxicol. 2015; 34(2): 162-181
[NPL 7] J. Ethnopharmacol. 2018; 224:261-272
[NPL 8] J. Cell. Biochem. 2019; 120(1): 425-438
[NPL 9] Acta Neuropathol. Commun. 2017; 5(1): 26, doi: 10.1186/s4 0478-017-0429-5

### SUMMARY OF INVENTION

### Technical Problem

An object of the present invention is to provide: a CD36 inhibitor which targets a subject (for example, a mammal) suffered from NASH, which has a risk of developing hepatocarcinoma; and a drug to prevent or treat NASH-derived hepatocarcinoma. The object thereof also includes to provide a prophylactic, therapeutic, and/or ameliorating drug or food composition for diseases other than the NASH-derived hepatocarcinoma pertaining to activation of CD36.

### Solution to Problem

Under the circumstances as described above, the inventors of the present invention carried out an extensive investigation; as a result, it was found that taxifolin or a pharmacologically acceptable salt thereof exhibited an excellent CD36 inhibition effect, and also found for the first time that these would become an effective, safe drug or food to prevent, treat, and/or ameliorate the NASH-derived hepatocarcinoma, for which there had been no effective prophylactic, therapeutic, or ameliorating method. The present invention has been accomplished on the basis of such finding. Namely, the present invention is as follows.
[1] A CD36 inhibitor for a mammal suffered from nonalcoholic steatohepatitis, comprising taxifolin or a pharmacologically acceptable salt thereof (hereinafter, this is sometimes referred to as “CD36 inhibitor according to the present invention”).
[2] A prophylactic or therapeutic drug for a disease pertaining to activation of CD36, in which the drug contains the inhibitor as described in [1] in a pharmacologically effective amount and is effective by administering to a mammal suffered from nonalcoholic steatohepatitis (hereinafter, this is sometimes referred to as “drug according to the present invention”).
[3] The drug as described in [2], in which the disease pertaining to activation of CD36 is hepatocarcinoma that is derived from nonalcoholic steatohepatitis.
[4] The drug as described in [2] or [3], in which the drug is prepared in the form of a tablet, a pill, a capsule, powders, grains, fine subtilaes, a troche, a liquid medicine, a nasal drop, or an injectable.
[5] The drug as described in any of [2] to [4], in which a pharmacologically effective amount of the drug per 1 kg body weight of a mammal per 1 day in terms of taxifolin is in the range of 0.2 to 200 mg.
[6] A food composition for a disease pertaining to activation of CD36, in which the composition contains the inhibitor as described in [1] in an effective amount and is effective by feeding to a mammal suffered from nonalcoholic steatohepatitis (hereinafter, this is sometimes referred to as “food composition according to the present invention”).
[7] The food composition as described in [6], in which the disease pertaining to activation of CD36 is hepatocarcinoma that is derived from nonalcoholic steatohepatitis.
[8] A method for inhibiting CD36 in a mammal, the method including administration of the inhibitor as escribed in [1] to the mammal in a pharmacologically effective amount.
[9] A method for treating or preventing hepatocarcinoma that is derived from nonalcoholic steatohepatitis, in which the method includes administering the inhibitor as described in [1] to a mammal in a pharmacologically effect amount.
[10] The method as described in [8] or [9], in which the pharmacologically effective amount per 1 kg body weight of a mammal per 1 day in terms of taxifolin is in the range of 0.2 to 200 mg.
[11] A prophylactic or ameliorating food composition for hepatocarcinoma that is derived from nonalcoholic steatohepatitis, in which the food composition contains taxifolin or a salt thereof in an effective amount.
[12] The food composition as described in [11], in which the food composition is attached with a display describing such as “use for lowering hepatocarcinoma risk” and/or “use for making hepatocarcinoma-resistant body”.
[13] The food composition as described in or [12], in which the food composition is prepared as a supplement, a food with functional claims, a health food, a food for special dietary uses, a food with health claims, a food for specified health uses, or a food with nutrient function claims.
[14] The food composition as described in any of to [13], in which the food composition is prepared in the form of a tablet, a pill, a capsule, powders, grains, fine subtilaes, a troche, a liquid formulation, a nasal tube feeding, or an enteral nutrition.
[15] Taxifolin or a pharmacologically acceptable salt thereof to prevent and/or treat hepatocarcinoma that is derived from nonalcoholic steatohepatitis, for a subject suffered from nonalcoholic steatohepatitis.
[16] Use of taxifolin or a pharmacologically acceptable salt thereof to produce a prophylactic and/or therapeutic drug for hepatocarcinoma that is derived from nonalcoholic steatohepatitis for a subject suffered from nonalcoholic steatohepatitis.

### Effects of Invention

According to the present invention, it is possible to provide an excellent CD36 inhibitor consisting of taxifolin that is derived from a safe plant or a pharmacologically acceptable salt thereof. The CD36 inhibitor according to the present invention can suppress the CD36 activity even with a low concentration, and also this is effective to a subject (e.g., a mammal) suffered from NASH, which may develop to hepatocarcinoma; thus, it is possible to effectively suppress the onset or development of the NASH-derived hepatocarcinoma. Therefore, according to the present invention, it is possible to provide a prophylactic, therapeutic, and/or ameliorating drug or food composition for the NASH-derived hepatocarcinoma, these drug and food composition containing, as the effective component, taxifolin or a pharmacologically acceptable salt thereof. According to the present invention, it is also possible to provide a prophylactic, therapeutic, and/or ameliorating drug or food composition for the diseases other than the NASH-derived hepatocarcinoma pertaining to activation of CD36, these drug and food composition containing, as the effective component, taxifolin or a pharmacologically acceptable salt thereof.

### BRIEF DESCRIPTION OF DRAWINGS

FIG. 1 describes the taxifolin's action on the CD36 gene expression in the liver tissue of the high-fat diet-induced model mouse.

FIG. 2 shows the taxifolin's action on the fatty acid capturing capacity in the HepG2 cell. (A) shows the taxifolin's action under the condition of a low glucose concentration (0.1%), (B) shows the taxifolin's action under the condition of a high glucose concentration (0.45%), and (C) shows the taxifolin's action in absence of glucose.

FIG. 3 shows the taxifolin's action on the development of hepatocarcinoma in the MC4R deficient mouse loaded with a high fat diet. (A) shows the taxifolin's action on the number of the precancerous symptoms, (B) shows the taxifolin's action on the number of the tumors having the precancerous symptom clustered, and (C) shows the taxifolin's action on the size of the tumor.

### DESCRIPTION OF EMBODIMENTS OF INVENTION

The terms used in this specification are defined as follows.

In this specification, “taxifolin” is one type of flavonoid, a compound represented by the formula (1), occupying most of the active components of Zigbertin (90% or more), which is a bioflavonoid composite that is separated from a wooden portion of Dahurian larch or of Siberia larch.

In this specification, the salt of taxifolin may be any salts that are known in this technical field, as far as it is not toxic. Specifically, it is a pharmacologically acceptable salt that may be readily formed by the action of a pharmacologically accepted base. Illustrative examples of the salt like this include an alkali metal salt such as a sodium salt and a potassium salt.

Taxifolin that is derived from Siberia larch is optically active ((+)-taxifolin). The taxifolin according to the present invention includes not only (+)-taxifolin, but also its enantiomer (−)-taxifolin, compounds with low optical purity (mixture of these enantiomers having high optical purities in an arbitrary ratio), and racemates. The taxifolin according to the present invention also includes labelled compounds thereof, i.e., those compounds having one, or two or more atoms that constitute the taxifolin labelled with an isotope element (e. g., ²H, ³H, ¹¹C, ¹³C, ¹⁴C, ¹⁵N, ¹⁸O, ¹⁸F, and ³⁵S).

The optically active taxifolin or a pharmacologically acceptable salt thereof may be produced by chemical synthesis with a publicly known method for it, or by extracting from and/or purifying a wooden portion of Dahurian larch or Siberian larch. Specifically, the optically active compound may be obtained by using an optically active synthesis intermediate, or conducting optical resolution of the finally obtained racemates by a normal method (e. g., see J. Jacques et al., “Enantiomers, Racemates and Resolution, John Wiley And Sons, Inc.”). (+)-Taxifolin is sold in a market, so that a commercially available product may be uses as it is.

Taxifolin or the pharmacologically acceptable salt thereof according to the present invention may be crystalline, in which a single crystal form or multiple crystal forms may be included in taxifolin or the salt thereof. The crystalline product may be produced by conducting crystallization using a publicly known crystallizing method for it.

Taxifolin or the pharmacologically acceptable salt thereof according to the present invention may include their solvates. These solvates are taxifolin or the pharmacologically acceptable salt thereof that have a solvent molecule coordinated thereto, including the hydrates thereof. Illustrative examples thereof include a hydrate, an ethanol adduct, and a dimethyl sulfoxide adduct of taxifolin or the pharmacologically acceptable salt thereof.

In this specification, the term “inhibits CD36” means disappearance or decrease of the activity of CD36; this means, for example, that gene expression or function of CD36 is inhibited under the conditions in Test Example 1 or Test Example 2 to be described later. The disappearance or decrease of the activity of CD36 is conducted preferably by the clinical indication of human.

“To Inhibit CD36” is preferably “to inhibit human CD36”.

“CD36 inhibitor” means the substance that inhibits expression of the CD36 gene. “CD36 inhibitor” is preferably “human CD36 inhibitor”.

In this specification, “disease pertaining to activation of CD36” means the disease that worsens sickness and/or symptom due to increase in the expression of the CD36 gene. Illustrative examples of the “disease pertaining to activation of CD36” include: malignant tumor such as NASH-derived hepatocarcinoma, breast cancer, prostate cancer, ovarian cancer, colon cancer, glioma, chronic myeloid leukemia, as well as NASH, hepatitis C, heart disease, stroke, age-related cardiomyopathy, kidney disease, diabetes, insulin resistance, arteriosclerotic disease, lipid metabolism disorders, metabolic syndrome, mastopathy, cerebral amyloid angiopathy, Alzheimer disease, thrombin-dependent coagulation, asthma, inflammatory bowel disease, epilepsy, Kawasaki disease, and mycobacterial infection. The “disease pertaining to activation of CD36” is preferably NASH-derived hepatocarcinoma.

In this specification, “prevention” includes suppression of disease onset.

In this specification, “treatment” includes amelioration of symptom, prevention of aggravation, maintenance of remission, prevention of flare-ups, secondary prevention, and prevention of recurrence.

In this specification, “prophylactic or therapeutic drug” includes a drug for prevention and/or treatment, or even a food composition for prophylactic and/or ameliorating use.

In this specification, “pharmacologically effective amount” means a dose of taxifolin or the pharmacologically acceptable salt thereof that is administered to the subject orally or non-orally (such as topical, rectal, intravenous, intramuscular, subcutaneous, and nasal).

In this specification, “subject” means the subject to which a drug or a food containing the effective component in an effective amount necessary to prevent and/or treat (or ameliorate) the disease or the sickness is administered or fed. Illustrative examples of the “subject” include mammals such as human, mouse, rat, hamster, guinea pig, rabbit, cat, dog, pig, cow, horse, sheep, and monkey; among these, human is preferable.

### Drug According to the Present Invention (or Drug Composition According to the Present Invention)

As can be seen in Test Examples to be described later, taxifolin can inhibit the activity of CD36 with a low concentration thereof, allowing for taxifolin or the pharmacologically acceptable salt thereof, or a drug containing these as effective components to be suitably used as the drug for the diseases pertaining to the activation of CD36, in particular, as the drug for prevention and/or treatment of the NASH-derived hepatocarcinoma.

The drug according to the present invention may be any of the drug consisting of taxifolin or the pharmacologically acceptable salt thereof and the drug composition that is obtained by blending taxifolin or the pharmacologically acceptable salt thereof with a pharmacologically acceptable supporting material or the like. The drug or the drug composition according to the present invention may be administered to the subject in a pharmacologically allowed effective amount thereof. Illustrative examples of the administering method of the drug or the drug composition according to the present invention include oral administration, intravenous administration, intraperitoneal administration, transdermal administration, intramuscular administration, intranasal administration, and mucosal administration. Among these, oral administration is preferable.

Illustrative examples of the pharmacologically acceptable support include excipients (such as starch, corn starch, lactose, Arabic gum, carboxymethyl cellulose, hydroxypropyl cellulose, and crystalline cellulose), lubricants (such as magnesium stearate, talc, and hardened rapeseed oil), disintegrants (such as carboxymethyl cellulose and talc), solvents (such as injection water, physiological salt water, Ringer's solution, alcohol, propylene glycol, polyethylene glycol, sesame oil, corn oil, olive oil, and cottonseed oil), dissolution aids (such as polyethylene glycol, propylene glycol, D-mannitol, trehalose, benzyl benzoate, ethanol, trisaminomethane, cholesterol, triethanolamine, sodium carbonate, sodium succinate, sodium salicylate, and sodium acetate), suspending agents (surfactants such as stearyl triethanolamine, sodium laurylsulfate, lauryl aminopropionate, lecithin, benzalkonium chloride, benzethonium chloride, and glycerin monostearate; coating substrates such as polyvinyl alcohol, polyvinyl pyrrolidone, sodium carboxymethylcellulose, methylcellulose, hydroxymethyl cellulose, hydroxyethyl cellulose, and hydroxypropyl cellulose; polysolvates; and polyoxyethylene-cured castor oil), isotonic agents (such as sodium chloride, glycerin, D-mannitol, D-sorbitol, and glucose), buffering agents (such as phosphate salts, acetate salts, carbonate salts, and succinate salts), thickeners (such as sodium alginate, xanthan gum, sodium chondroitin sulfate, polyvinyl alcohol, and povidone), preservatives (such as p-oxybenzoate esters, chlorobutanol, benzyl alcohol, phenethyl alcohol, dehydroacetic acid, and sorbic acid), antioxidants (such as sulfite salts, ascorbic acid salt (Vitamin C), and tocopherol (Vitamin E)), colorants (such as water-soluble, edible tar pigments (edible pigments such as edible red No. 2 and No. 3, edible yellow No. 4 and No. 5, and edible blue No. 1 and No. 2), water-insoluble lake pigments (such as aluminum salts of the above-mentioned water-soluble, edible tar pigments), and naturally occurring pigments (such as B-carotin, chlorophyll, and bengala)), sweeteners (such as saccharin sodium, dipotassium glycyrrhizinate, aspartame, and stevia), and other components (such as larch-forming layers and wood extracts).

After mixing with the components described above, the drug composition according to the present invention may be formulated in accordance with a publicly known procedure into a tablet, a pill, a capsule (including a hard capsule, a soft capsule, and a microcapsule), powders, grains, fine subtilaes, a troche, a liquid medicine (including syrup, emulsion, and suspension), a nasal drop, and an injectable.

With regard to the drug composition in the form of a tablet, grains, and fine subtilaes, in view of masking the taste, improving photo-stability, appearance, and enteric property, the composition may be coated by a publicly known method using the afore-mentioned coating substrates.

There is no particular restriction in the content of taxifolin or the pharmacologically acceptable salt thereof in the drug according to the present invention; for example, the content is 1% by weight or more and 100% by weight or less relative to total weight of the formulation, although these values vary depending on the form of the formulation. The lower limit value thereof is preferably 5% by weight or more, more preferably 30% by weight or more. The upper limit value thereof is preferably 100% by weight or less, more preferably 95% by weight or less.

There is no particular restriction in the dose (i.e., pharmacologically effective amount) of taxifolin or the pharmacologically acceptable salt thereof; for example, in the case of dosing to a human adult patient by oral administration, the dose per body weight of 1 kg and per one day in terms of taxifolin, i.e., the effective component, is 0.001 mg/kg/day or more and 200 mg/kg/day or less, although these values vary depending on the subject to be administered, disease, sickness, formulation, administration route, among other things. The lower limit value thereof is preferably 0.01 mg/kg/day or more, more preferably 0.1 mg/kg/day or more, still more preferably 0.2 mg/kg/day or more. On the other hand, the upper limit value thereof is preferably 50 mg/kg/day or less, more preferably 10 mg/kg/day or less.

There is no particular restriction in the number of administration of taxifolin or the pharmacologically acceptable salt thereof; for example, it may be three times per day, twice per day, once per day, every two days, or even every three days. The administration may be made any time, i.e., before meal, after meal, or between meals.

There is no particular restriction in the administration period of taxifolin or the pharmacologically acceptable salt thereof.

Taxifolin or the pharmacologically acceptable salt thereof may be used in combination with other treatments (for example, chemotherapy, operations (such as surgical resection, radiofrequency ablation, and hepatic artery embolization), and radiation therapy (such as X-ray and particle beam therapy). By so doing, the prophylactic effect and/or therapeutic effect of taxifolin or the pharmacologically acceptable salt thereof may be further enhanced. Among these, a combination with chemotherapy is especially preferable. Specific embodiments of the combination with chemotherapy will be described below.

Taxifolin or the pharmacologically acceptable salt thereof may be used together with other drug (concomitant drug) as far as the medical effect thereof is not impaired. Here, there is no restriction in the administration timing; they may be administered simultaneously, or at the different timing. In addition, they may also be administered as a single formulation in a combination of taxifolin or the pharmacologically acceptable salt thereof with the concomitant drug.

The dose of the concomitant drug may be chosen as appropriate with reference to the clinically used dose thereof. The mixing ratio of taxifolin or the pharmacologically acceptable salt thereof with the concomitant drug may be chosen as appropriate depending on the administering subject, the administering route, the subject disease, the sickness, the type of the concomitant drug, among other thing.

When using together with taxifolin or the pharmacologically acceptable salt thereof for prevention and/or treatment of the NASH-derived hepatocarcinoma, illustrative examples of the concomitant drug include a molecularly targeting drug, an immune checkpoint inhibitor, a chemotherapeutic agent, and a hepatoprotective drug.

Illustrative examples of the “molecularly targeting drug” include: EGFR inhibitors such as afatinib, erlotinib, gefitinib, cetuximab, and panitumumab; ALK inhibitors such as crizotinib; EGFR/HER2 inhibitors such as lapatinib; HER2 inhibitors such as trastuzumab, trastuzumab emtansine, and pertuzumab; angiogenesis inhibitors such as bevacizumab, axitinib, sunitinib, sorafenib, pazopanib, regorafenib, lenvatinib, and ramucirumab; mTOR inhibitors such as everolimus and temsirolimus; BCR-ABL inhibitors such as imatinib, dasatinib, and nilotinib; membrane differentiation antigen targeting agents such as ibritumobab tiuxetan, ofatumumab, rituximab, brentuximab vedotin, gemzumab ozogamicin, and mogamulizumab; denosumab; and bortezomib.

Illustrative examples of the “immune checkpoint inhibitor” include: anti-PD-1 antibodies such as nivolumab and pembrolizumab; anti-CTLA-4 antibodies such as ipilimumab; and anti-PD-L1 antibodies such as avelumab, atezolizumab, and durvalumab.

Illustrative examples of the “chemotherapeutic agent” include: alkylating agents such as busulfan, cyclophosphamide, dacarbazine, ifosfamide, melphalan, nimustine hydrochloride, procarbazine hydrochloride, ranimustine, temozolomide, and thiotepa; antimetabolites such as fluorouracil, mercaptopurine hydrate, cladribine, carmofur, cytarabine ocfosfate hydrate, cytarabine, doxifluridine, enocitabine, fludarabine phosphate, gemcitabine hydrochloride, hydroxycarbamide, methotrexate, nelarabine, pemetrexed sodium hydrate, pentostatin, tegafur, tegafur/uracil combination, and tegafur/gimeracil/oteracil combination; vinca alkaloid type drugs such as vinblastine hydrochloride, vincristine hydrochloride, vindesine hydrochloride, and vinorelbine tartrate; taxane type drugs such as docetaxel hydrate and paclitaxel; antitumor antibiotics such as aclarubicin hydrochloride, actinomycin D, amrubicin hydrochloride, bleomycin hydrochloride, daunorubicin hydrochloride, doxorubicin hydrochloride, epirubicin hydrochloride, idarubicin hydrochloride, mitomycin C, mitoxantrone hydrochloride, peplomycin hydrochloride, and pirarubicin hydrochloride; topoisomerase inhibitors such as etoposide, irinotecan hydrochloride hydrate, nogitecan hydrochloride, and sobuzoxane; platinum-containing drugs such as carboplatin, cisplatin, nedaplatin, and oxaliplatin; hormone drugs such as anastrozole, exemestane, letrozole, tamoxifen citrate, toremifene citrate, fadrozole hydrochloride hydrate, estramustine phosphate sodium, flutamide, goserelin acetate, leuprorelin acetate, medroxyprogesterone acetate, and mepitiostane; anti-malignant tumor streptococcal drugs such as pisibanil; aceglatone, arsenic trioxide, coriolus versicolor polysaccharide drug powder, L-asparaginase, lentinan, levofolinate, mitotane, porfimer sodium, schizophyllan, tamibarotene, tretinoin, and ubenimex.

Illustrative examples of “hepatoprotective drug” include ursodeoxycholic acid, glycyrrhizin formulation, minor bupleurum decoction, taurine, stronger neo-minophagen C, glutathione, and diisopropylamine dichloroacetate.

In a certain embodiment, the CD36 inhibitor according to the present invention, or the drug containing this (or drug composition) may be provided in the form of a kit (administration, treatment, and/or prevention kit), a package (packaging and so forth), or a drug set (and/or a bottle), together with a written document describing whether this may be or must be used for treatment and/or prevention. The kit, the package, and the drug set as described above may be provided with one or more containers containing the CD36 inhibitor and/or other drug or medicine (or ingredient). Examples of the kit, the package, and the drug set as described above include a commercial kit, a commercial package, and a commercial drug set that are appropriately prepared for treatment and/or prevention of the target disease. Examples of the written document included in the kit, the package, and the drug set as described above include a caution note or an attachment document that is written in the form specified by a governmental organization that control production, use, or sales of a drug or a biological product, and this includes a caution note or an attachment document indicating approval of production, use, or sales of the product pertaining to administration to human, issued from the relevant government organization. The kit, the package, and the drug set as described above include an enveloped product, and they may also include a structure that is configured to conduct proper administering steps, or a structure that is configured to achieve further preferable medical treatment and/or prevention, including treatment and/or prevention of a targeted disease.

### Food Composition According to the Present Invention

The food composition according to the present invention may be any of the one that is composed of only taxifolin or the pharmacologically acceptable salt thereof and a mixture of taxifolin or the pharmacologically acceptable salt thereof with a food additive and so forth. There is no particular restriction in the type, form, or the like of the food composition as far as it contains taxifolin or the salt thereof and may be orally ingested by a subject. As can be seen in Test Examples to be described later, taxifolin is able to exhibit an excellent CD36 inhibition action to the subject suffered from NASH; thus, the food composition according to the present invention may be used for prophylactic and/or ameliorating use in the NASH-derived hepatocarcinoma.

Illustrative examples of the food composition according to the present invention include: confectioneries such as troche, drop, candy, ramune, gummy, and chewing gum; western confectioneries such as cooky, cracker, biscuit, potato chips, bread, cake, chocolate, doughnut, pudding, and jelly; Japanese confectioneries such as fired bread, yokan, daihuku, ohagi, sweet bun, and castella; frozen desserts such as ice cream, ice candy, sherbet, and gelato; breads such as plain bread, French bread, and croissant; noodles such as udon (Japanese noodle), soba, Chinese noodle, and kishimen; fish paste products such as kamaboko, chikuwa, and fish sausage; animal meat products such as ham, sausage, hamburger, and corned beef; seasonings such as salt, pepper, miso, soy sauce, sauce, dressing, mayonnaise, ketchup, sweeteners (such as sugar, honey, powdered candy, starch syrup, jam, and marmalade), and spices (such as mustard and pepper); teppanyaki foods (foods cooked on a hot plate) such as Akashi-yaki, tako-yaki (octopus balls), okonomi-yaki (Japanese pancake), monjya-yaki (formless version of okonomi-yaki), yakisoba, and yakiudon; milk products such as cheese, butter, margarin, and yogurt; various side dishes such as natto (tempered soybean), tofu (bean curd), atsu-age (fried bean curd), konjac, dumpling, pickles, tsukudani (food boiled in soy sauce), gyoza (Chinese dumpling), shumai (another kind of gyoza), croquette, sandwich, pizza, hamburger, and salad; animal products such as beef, pork, and chicken; sea products such as shrimp, scallop, freshwater clam, and kelp; various powdered products of vegetable, fruit, plant, yeast, and algae; powdered and solidified oil and fats/flagrances (vanilla, citrus, and tuna); and drinks.

Illustrative examples of the drink include: drinking food products such as soup and miso soup; powdered food products such as instant coffee, instant tea, instant milk, instant soup, and instant miso soup; alcoholic drinks such as whisky, bourbon, spirits, liqueur, wine, fruit wine, Japanese sake, Chinese liquor, shochu (Japanese version of vodka), beer, non-alcoholic beer with 1% or less of alcohol degree, low-malt beer, and chu-high (shochu mixed with soda water); non-alcoholic drinks such as drinks containing fruit juice (such as juice of apple, orange, grape, banana, peach, and plum), drinks containing vegetable juice (such as juice of tomato, carrot, celery, cucumber, and water melon), drinks containing fruit juice and vegetable juice, soft drinks, cow milk, soy milk, dairy drinks, drinkable yogurt, coffee, cocoa, tea drinks (such as black tea, green tea, wheat tea, brown rice tea, sencha (Japanese roasted green tea), gyokuro tea (high quality Japanese green tea), hojicha (another type of Japanese roasted green tea), oolong tea, turmeric tea, pu-erh tea, rooibos tea, rose tea, chrysanthemum tea, herb tea (such as mint tea and jasmine tea)), energy drinks, sports drinks, and mineral water.

Illustrative examples of the food composition preferable include jelly, tea drinks, alcoholic drinks, drop, candy, ramune, cooky, cracker, biscuit, chocolate, cheese, butter, margarine, and chewing gum.

The food composition according to the present invention may be prepared as the food with functional claims, the health food, the food for specified health uses, the food for special dietary uses (foods for sick people such as a hospital food, a patient food, and a supporting food), or a supplement; it is preferable to be prepared as the food for specified health uses, the food for special dietary uses, or a supplement.

Illustrative examples of the form of the food composition according to the present invention include a tablet, a pill, a capsule (including a hard capsule, a soft capsule, and a microcapsule), powders, grains, fine subtilaes, a troche, a liquid formulation (including syrup, emulsion, and suspension), a nasal tube feeding, and an enteral nutrition; among these, a tablet and a pill are preferable.

The food composition according to the present invention is preferably the food for specified health uses, the food for special dietary uses, specific health food, or a supplement, in the form of a tablet or a capsule, in particular.

In this specification, the supplement means not only a nutrient assisting food, a food with nutrient function claims, etc., but also a health-assisting food, a food with health claims, etc., having, among others, functions to help maintenance, recovery, promotion, etc. of health (especially, the prophylactic function for hepatocarcinoma).

The food composition according to the present invention may be produced using a publicly known method, for example, by adding taxifolin or the salt thereof into food. Specifically, for example, the food composition in the form of a tablet may be produced, for example, by mixing taxifolin or the salt thereof with, among other things, materials such as an excipient (for example, lactose, sugar, mannitol, and corn starch), a sweetener, and a fragrance, followed by applying a pressure to the resulting mixture using a tableting machine to shape into a tablet. As needed, other materials (for example, additives including vitamins such as Vitamin C, minerals such as iron, a food fiber, a crystalline cellulose, a rapeseed (hardened rapeseed), and a larch forming layer/wooden portion extract) may be added. The food composition in the form of a capsule may be produced, for example, by filling a food composition containing taxifolin or the salt thereof in the form of liquid, suspension, paste, powder, or grain into a capsule, or by molding said composition with a covering that uses a capsulating substrate.

In addition to a food material, a food additive, various nutrients, a vitamin, a flavor (such as cheese or chocolate), etc., which are usually used in this field, the food composition according to the present invention may be added by, among other things, a physiologically acceptable supporting material as long as this will not impair the advantageous effects of the present invention. Examples of the physiologically acceptable supporting material include customarily used various organic or inorganic supporting materials; and illustrative examples thereof include an excipient, a binder, a disintegrant, a lubricant, a colorant, a sweetener, a preservative, an antioxidant, a thickener, and an emulsifier. Examples of the food additive include a colorant, a sweetener, a preservative, an antioxidant, and a flavor. In addition, other materials such as minerals including iron and calcium, as well as food fibers such as pectin, carrageenan, and mannan may also be included.

Illustrative examples of each of the excipient, the binder, the disintegrant, the lubricant, the solvent, the dissolution aid, the suspending agent, the buffering agent, the thickener, the colorant, the sweetener, the preservative, and the antioxidant may be the same as those that are used for the drug according to the present invention, as described before.

The vitamins may be any of water-soluble and fat-soluble. Illustrative examples thereof include retinol palmitate, tocopherol (Vitamin E), bisbentiamine, riboflavin, pyridoxine hydrochloride, cyanocobalamin, sodium ascorbate (Vitamin C), cholecalciferol, nicotinamide, calcium pantothenate, folic acid, biotin, choline bitartrate, niacin, and nicotinamide mononucleotide (NMN).

With regard to the food composition in the form of a tablet, grains, and fine subtilaes, in view of masking the taste, improving photo-stability, appearance, or enteric property, the composition may be coated by a publicly known method using a coating substrate. The coating substrate may be the same as those used for the drug according to the present invention, as described before; the same is true for the method for coating.

There is no particular restriction in the content of taxifolin or the salt thereof in the food composition according to the present invention; the content is, for example, 0.1% by weight or more and 50% by weight or less, relative to total weight of the food composition. The lower limit value thereof is preferably 0.5% by weight or more, more preferably 1.0% by weight or more. On the other hand, the upper limit value thereof is preferably 20% by weight or less, more preferably 10% by weight or less.

Because the food composition obtained as described above is safe, this may be continuously fed to a subject, preferably to human, in particular.

There is no particular restriction in the intake of the food composition according to the present invention as far as it is within the effective amount for taxifolin or the salt thereof to inhibit CD36 or to prevent and/or ameliorate the NASH-derived hepatocarcinoma. For example, in the case of feeding the food composition according to the present invention to a human adult with the purpose to prevent and/or ameliorate the NASH-derived hepatocarcinoma, the intake of taxifolin or the salt thereof per body weight of 1 kg and per one day in terms of taxifolin, i.e., the active component, is 0.001 mg/kg/day or more and 200 mg/kg/day or less, although these values vary depending on the subject to be fed, the feeding form, the feeding amount, among others. The lower limit value thereof is preferably 0.01 mg/kg/day or more, more preferably 0.1 mg/kg/day or more, still more preferably 0.2 mg/kg/day or more. On the other hand, the upper limit value thereof is preferably 50 mg/kg/day or less, more preferably 10 mg/kg/day or less. Also, from the viewpoint to express the advantageous effects without adversely affecting palatability and intake amount, the intake amount as described above is preferable. In the case that the subject is other mammals, the same intake amount is applicable.

There is no particular restriction in the number of feeding of taxifolin or the pharmacologically acceptable salt thereof; for example, it may be three times per day, twice per day, once per day, every two days, or even every three days. The administration may be made any time, i.e., before meal, after meal, or between meals.

The food composition according to the present invention may be used by itself, but it may also be used in combination with other treatments (i.e., chemotherapy, operation, and radiation therapy). Specifically, a combination with a drug composition, a food composition, or a feedstuff, having an ameliorating action on other liver functions, may be used. When combined with other treatments, the prophylactic effect and/or ameliorating effect on hepatocarcinoma may be further enhanced.

The food composition according to the present invention includes those that are classified into the health food, the food with functional claims, the food for specified health uses, the food with health claims, the food having the display of reduction in a disease risk, or the food for special dietary uses (foods for sick people such as a hospital food, a patient food, and a supporting food). The display of reduction in a disease risk may include, such as for example, “For risk reduction of hepatocarcinoma” and “Create the hepatocarcinoma-resistant body”. Therefore, the food composition according to the present invention is the food and drink having the attached display, expressing that, for example, said food composition contains taxifolin or the salt thereof, thereby lowering a disease risk of hepatocarcinoma.

The functions of these food compositions may be displayed on any of a product main body, a container, and a package, as well as may be written in an explanation document, an attached document, or a campaign document.

### EXAMPLES

Hereinafter, the present invention will be described in more detail on the basis of Test Examples and Formulation Examples. However, the present invention is not limited by Test Examples and Formulation Examples, so that this may be changed within the range not deviating from the present invention. The reagents, equipment, materials, among other things, that are used in the present invention are commercially available unless otherwise specifically noted. The percentage (%) means “% by weight” unless otherwise specifically noted.

The taxifolin in Test Examples below was purchased from Ametis JSC (Russia) and used as it was.

### Test Example 1: Suppression Effect of Taxifolin on CD36 Gene Expression

In Test Example 1, the taxifolin's action on the CD36 gene expression in the liver tissue was studied using the high-fat diet-induced model mice.

### (Mice Breeding and Intake Experiments)

The C57BL/6J male mice (7 weeks old: CLEA Japan, Inc.) were acclimated for one week with a normal solid diet (MF: Oriental Yeast Co., Ltd.) and with freely available drinking water.

The 8 weeks old mice after the acclimation were divided into 4 groups with 5 mice each, namely, the group of those raised with a normal solid diet for 12 weeks until 20 weeks old (hereinafter, “ND Group”), the group raised with a high fat diet (HFD-60 (506 kcal/100 g; 60% fat content in terms of energy rate): Oriental Yeast Co., Ltd.) (hereinafter, “HFD Group”), the group raised with the high fat diet containing 1% by weight of taxifolin (hereinafter, “HFD+1% TX-administered Group”), and the group raised with the high fat diet containing 3% by weight of taxifolin (hereinafter, “HFD+3% TX-administered Group”).

### (Preparation of Measurement Sample)

The mice raised until 20 weeks old were anesthetized with ether, and after they were euthanized by cervical dislocation, the liver of each individual was removed.

The resulting removed liver was cut into a cuboid with one side of about 2 to 3 mm; and from this, total RNA was extracted by using the RNA extraction kit (RNeasy Mini kit: QIAGEN K.K.). Using the total RNA thus extracted, cDNA was synthesized by the reverse transcription reaction to obtain a measurement sample.

### (Measurement of the Amount of CD36 Gene Expression)

By using the resulting measurement sample, the real-time PCR was conducted using the real-time PCR instrument (SYBR Green Realtime PCR Master Mix: Thermo Fisher Scientific Inc.).

The mRNA expression amount of CD36 was corrected by the expression amount of glyceraldehyde-3-phosphate dehydrogenase (GAPDH), and this was comparatively quantified using the ΔΔCt method. The primers used in the PCR are listed in Table 1 below.

TABLE 1

| Sequence name | Sequence | |
|---|---|---|
| CD36 forward primer | aatggcacagacgcagcct | Sequence number 1 |
| CD36 reverse primer | ggttgtctggattctgga | Sequence number 2 |
| Gapdh forward primer | agtatgactccactcacggcaa | Sequence number 3 |
| Gapdh reverse primer | tctcgctcctggaagatggt | Sequence number 4 |

### (Statistical Analysis)

The statistical analysis was conducted using Student t-test.

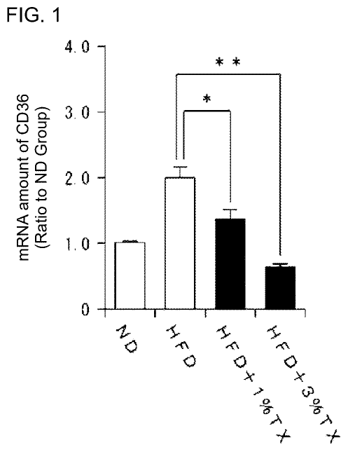

In FIG. 1, the mRNA expression amount of CD36 is expressed by the average value of each group±standard error. When the P-value is less than 0.05, it is statistically judged to be significantly different and marked with “*”, and when the P-value is less than 0.001, the mark “**” is attached.

### Result 1

FIG. 1 shows the mRNA amount of CD36 in the liver tissue of the mouse after 12 weeks from the start of feeding test in each group.

As can be seen in FIG. 1, in the HFD Group loaded with the high fat diet, it was recognized that the mRNA expression amount of CD36 in the liver tissue had significantly increased as compared with the ND Group that is fed with a normal solid diet. The increase in the mRNA expression amount of CD36 due to the load with the high fat diet is significantly decreased in the HFD+1% TX-administered Group and in the HFD+3% TX-administered Group, depending on the taxifolin dose. Especially, in the HFD+3% TX-administered Group, the mRNA expression amount of CD36 was suppressed to the same level as the ND Group fed with the normal solid diet, keeping the normal value.

Accordingly, it became clear that taxifolin remarkably suppressed the increase in the CD36 gene expression due to obesity.

### Test Example 2: Taxifolin's Suppressing Action on Fatty Acid Capturing

In Test example 2, the taxifolin's action on fatty acid capturing was studied by using the HepG2 cell, which is the cell strain derived from human hepatocarcinoma.

### (Cell Culture and Fatty Acid Capturing Experiments)

The HepG2 cells were inoculated with the density of 0.2×10⁶ cells/well on the plate having 24 wells, and they were cultured using the 10% FBS-containing DMEM medium until the HepG2 cells covered 70 to 80% of the surface in the culture vessel. The HepG2 cells cultured in the culture medium were washed with the phosphate buffer (PBS), then cultured for 24 hours in the serum-free medium containing 400 μm of palmitic acid, taxifolin with various concentrations, and 1% BSA, with n=6 in each Group A to E, as described in Table 2.

The glucose concentration contained in the culture medium and in the serum-free medium were made 0%, 0.1% (low glucose), or 0.45% (high glucose).

TABLE 2

| | Culture medium | | Serum-free medium | | |
|---|---|---|---|---|---|
| | (10% FBS-containing DMEM) | | (1% BSA-containing DMEM) | | |
| Group | Palmitic acid | Taxifolin | Palmitic acid | Taxifolin | |
| A | − | − | − | − | |
| B | − | − | + | − | |
| C | − | − | + | 0.1 | nM |
| D | − | − | + | 0.1 | μM |
| E | − | − | + | 1 | μM |

### (Lipid Droplet Staining and Measurement)

The HepG2 cells that had been cultured in the serum-free medium for 24 hours were immobilized by 4% paraformaldehyde after washing with PBS. The resulting immobilized HepG2 cells were treated with 60% isopropanol, and then the lipid drops in the cells were stained using Oil Red O (Wako Pure Chemical Industries, Ltd.). Thereafter, it was washed with 60% isopropanol and then with PBS. The resulting washed HepG2 cells were treated with 100% isopropanol to elute the Oil Red O out. The resulting eluted Oil Red O was recovered in the plate with 96 wells, and then the absorbance (550 nm) was measured using the microplate reader (Multiscan SkyHigh; Thermo Fisher Scientific, Inc.).

### Statistical Analysis

The statistical analysis was conducted using Student t-test.

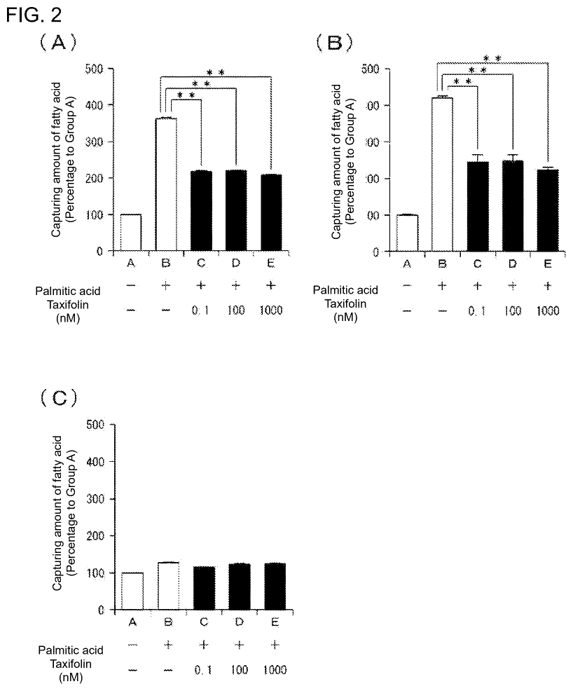

In FIG. 2, the capturing amount of the fatty acid is expressed by the average value of each group±standard error. When the P-value is less than 0.01, it is statistically judged to be significantly different and marked with “**”.

### Results 2

FIG. 2 indicates the capturing amount of the fatty acid in the HepG2 cells in each Group. The capturing amount in the HepG2 cells in each Group A to E is expressed by the percentage relative to Group A.

As can be seen in FIG. 2A, under the low glucose condition, in the Group B added with palmitic acid (i.e., fatty acid) in the medium, the eminent increase in the capturing amount of palmitic acid was recognized as compared with the Group A (i.e., reference group). On the other hand, the capturing amount of palmitic acid by the HepG2 cells was significantly decreased when taxifolin was added in an amount of 0.1 nM or more. As can be seen in FIG. 2B, there was the same tendency recognized in the taxifolin's action on the capturing amount of palmitic acid by the HepG2 cells under the high glucose condition.

Accordingly, it became clear that taxifolin suppressed the fatty acid capturing in the liver cells even with an amount of such a low concentration of 0.1 nM.

On the other hand, in the absence of glucose, there was no palmitic acid capturing recognized in the HpG2 cells (FIG. 2C).

The fatty acid capturing capacity of the HepG2 cell is dependent on the glucose outside the cells, suggesting involvement of ATP here.

When the fatty acid concentration outside the cell increases, CD36, which is the representative molecule to capture the fatty acid, is mobilized to the cell surface, resulting in activation of the fatty acid capturing capacity. The migration of CD36 to the cell membrane is complemented by the ATPase having the ATP pocket.

Accordingly, it is presumed that the taxifolin's suppression action on the fatty acid capturing in the liver cell is caused by inhibition of the CD36 function.

According to the results 1 and 2 above, taxifolin suppresses not only the CD36 gene expression but also the CD36 function that has already been expressed; thus, the CD36 inhibitor according to the present invention can provide an excellent method for inhibiting the CD36 activity.

### Test Example 3: Prophylactic Effect of Taxifolin on Hepatocarcinoma in MC4R Deficient Mouse

In Test Example 3, the taxifolin's action on the hepatocarcinoma by using the melanocortin-4 receptor deficient mouse (hereinafter, “MC4R deficient mouse”) loaded with a high fat diet.

### (Preparation of MC4R Deficient Mouse)

On the basis of the description in JP 2020-065454 A and JP 2017-006022 A, the MC4R deficient mouse provided by Joel K. Elmquist at the Medical Center in Texas University was used (Cell, 2005; 123(3): 493-505). The genetic background of the MC4R deficient mouse is the C57BL/6J mouse.

These mice were kept under the controlled condition of temperature, humidity, and lighting time (12 hours cycle of dark and bright), unless otherwise specifically noted. After weaning (4 weeks after the birth), they were raised with a high fat diet (D12492 (524 kcal/100 g; 60% fat content in terms of energy rate); Research Diets, Inc.) and with freely available drinking water.

### (Hepatocarcinoma Induction Experiment)

All the MC4R mice loaded with the high fat diet had the NASH-like symptom by 20 weeks old; it has been known that they develop hepatocarcinoma thereafter (Am. J. Pathol., 2011; 179(5):2454-2463).

The MC4R deficient 20 weeks old mice loaded with the high fat diet were divided into two groups; the reference group (n=13) that is continued to be fed with the same high fat diet until 50 weeks old, and the taxifolin-administered group (n=12) that is fed with the high fat diet containing 3% of taxifolin.

### (Measurement of Hepatocarcinoma Symptom)

The mice raised until 50 weeks old were euthanized by deep anesthesia using three mixed anesthetic agents, and then, the liver of each individual was removed.

The number of the precancerous symptoms (Foci) in the removed liver, the number of the tumors having the precancerous symptom clustered, and the size of the tumor were measured.

### Statistical Analysis

The statistical analysis was conducted using Student t-test.

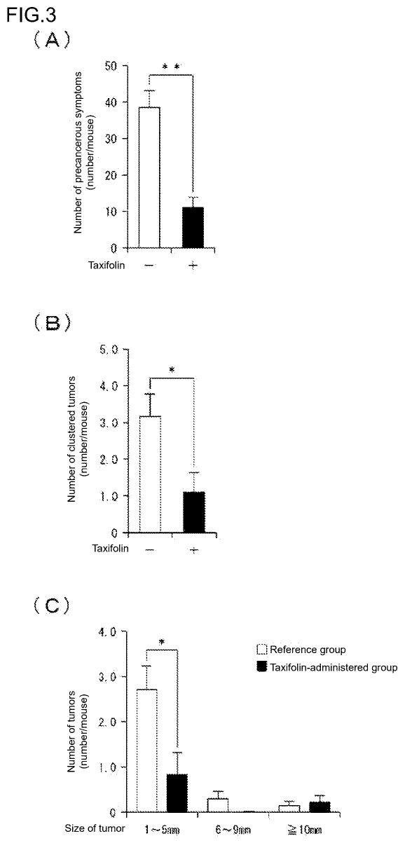

In FIG. 3, the number of the precancerous symptoms, the number of the tumors having the precancerous symptom clustered, and the size of the tumor are expressed by the average value of each group±standard error. When the P-value is less than 0.05, it is statistically judged to be significantly different and marked with “*”, and when the P-value is less than 0.001, the mark “**” was attached.

### Results 3

FIG. 3 shows the number of the precancerous symptoms, the number of the tumors having the precancerous symptom clustered, and the size of the tumor, in the reference group and the taxifolin-administered group.

As can be seen in FIG. 3A, in the taxifolin-administered group, the number of the precancerous symptoms decreased drastically as compared with the reference group. As can be seen in FIG. 3B, in the taxifolin-administered group, too, the number of the tumors having the precancerous symptom clustered decreased drastically as compared with the reference group.

Also, as can be seen in FIG. 3C, in the taxifolin-administered group, the size of the tumor between 1 and 5 mm decreased significantly as compared with the reference group.

Accordingly, it became clear that taxifolin suppresses the development of the NASH-related hepatocarcinoma.

According to the results 3 as described above, taxifolin is confirmed to suppress the development of the NASH-related hepatocarcinoma, so that the CD36 inhibitor according to the present invention can provide the drug and the food composition in order to prevent, treat, or ameliorate the NASH-related hepatocarcinoma.

Examples of the formulation according to the present invention include following, though the present invention is not limited to these.

### Formulation Example 1 (Production of Tablet Formulation)

| | | | |
|---|---|---|---|
| 1) taxifolin | | 50 | g |
| 2) corn starch | | 50 | g |
| 3) crystalline cellulose | | 40 | g |
| 4) hydroxypropyl cellulose | | 17 | g |
| 5) magnesium stearate | | 3 | g |
| 1000 tablets | total | 160 | g |

Whole of 1), 2), and 3) are kneaded with 12 g of 4); then, after dried under vacuum, the resulting powders are sorted. The powders thus sorted are mixed with 5 g of 4) and 3 g of 5), then, the mixture is tableted using a tableting machine to obtain 1000 tablets containing 50 mg of taxifolin in each tablet.

### Formulation Example 2 (Production of Tablet Formulation)

| | | | |
|---|---|---|---|
| 1) taxifolin | | 18 | g |
| 2) starch | | 100 | g |
| 3) crystalline cellulose | | 62 | g |
| 4) mannitol | | 15 | g |
| 5) Vitamin C | | 50 | g |
| 6) magnesium stearate | | 5 | g |
| 1000 tablets | total | 250 | g |

Whole of 1) and 2), and 40 g of 3) are kneaded with water; then, after dried under vacuum, the resulting powders are sorted. The powders thus sorted are mixed with whole of 4), 5) 6), and 22 g of 3), then, the mixture is tableted using a tableting machine to obtain 1000 tablets containing 18 mg of taxifolin in each tablet.

### Formulation Example 3 (Production of Tablet Formulation)

| | | | |
|---|---|---|---|
| 1) taxifolin | | 18 | g |
| 2) starch | | 100 | g |
| 3) crystalline cellulose | | 62 | g |
| 4) hardened rapeseed oil | | 15 | g |
| 5) Vitamin C | | 50 | g |
| 6) magnesium stearate | | 5 | g |
| 1000 tablets | total | 250 | g |

Whole of 1) and 2), and 40 g of 3) are kneaded with water; then, after dried under vacuum, the resulting powders are sorted. The powders thus sorted are mixed with whole of 4), 5), 6), and 22 g of 3), then, the mixture is tableted using a tableting machine to obtain 1000 tablets containing 18 mg of taxifolin in each tablet.

### Formulation Example 4 (Production of Capsule Formulation)

| | | |
|---|---|---|
| 1) taxifolin | | 50 mg |
| 2) crystalline cellulose | | 10 mg |
| 3) lactose | | 20 mg |
| | total | 80 mg |

1), 2), and 3) are mixed and filled into a gelatin capsule.

### Formulation Example 5 (Production of Capsule Formulation)

| | | | |
|---|---|---|---|
| 1) taxifolin | | 50 | mg |
| 2) corn starch | | 20 | mg |
| 3) lactose | | 30 | mg |
| | total | 100 | mg |

1), 2), and 3) are mixed and filled into a gelatin capsule.

### INDUSTRIAL APPLICABILITY

According to the present invention, it is possible to provide an excellent CD36 inhibitor consisting of taxifolin that is derived from safe plants or a pharmacologically acceptable salt thereof. The CD36 inhibitor according to the present invention can suppress the CD36 activity even with a low concentration, and also this is effective to the subject (e. g., a mammal) suffered from NASH, which may develop to hepatocarcinoma; thus, it is possible to effectively suppress the onset or development of the NASH-derived hepatocarcinoma. Therefore, according to the present invention, it is possible to provide a prophylactic, therapeutic, and/or ameliorating drug or food composition for the NASH-derived hepatocarcinoma, these drug and food composition containing, as the effective component, taxifolin or a pharmacologically acceptable salt thereof. According to the present invention, it is also possible to provide a prophylactic, therapeutic, and/or ameliorating drug or food composition for the diseases pertaining to activation of CD36 other than the NASH-derived hepatocarcinoma, these drug and food composition containing, as the effective component, taxifolin or a pharmacologically acceptable salt thereof.

This application is based on Japanese Patent Application No. 2022-100812 filed on Jun. 23, 2022 in Japan, and all the contents thereof are included in this specification.

1. A CD36 inhibitor for a mammal suffered from nonalcoholic steatohepatitis, comprising taxifolin or a pharmacologically acceptable salt thereof.

2. A prophylactic or therapeutic drug for a disease pertaining to activation of CD36, wherein the drug contains the inhibitor according to claim 1 in a pharmacologically effective amount and is effective by administering to a mammal suffered from nonalcoholic steatohepatitis.

3. The drug according to claim 2, wherein the disease pertaining to activation of CD36 is hepatocarcinoma that is derived from nonalcoholic steatohepatitis.

4. A food composition for a disease pertaining to activation of CD36, wherein the said composition contains the inhibitor according to claim 1 in an effective amount and is effective by feeding to a mammal suffered from nonalcoholic steatohepatitis.

5. The food composition according to claim 4, wherein the disease pertaining to activation of CD36 is hepatocarcinoma that is derived from nonalcoholic steatohepatitis.
